(19) United States
(12) Patent Application Publication
(10) Pub. No.: US 2011/0023154 A1
(43) Pub. Date: Jan. 27, 2011

(54) SILKWORM GENOME EDITING WITH ZINC FINGER NUCLEASES

(75) Inventors: Joseph Bedell, St. Louis, MO (US); Brian Buntaine, St. Louis, MO (US); Xiaoxia Cui, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,269

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A01K 67/04; C12N 5/07; C12N 5/10

(52) U.S. Cl. 800/13; 435/348

(57) ABSTRACT

The present invention provides a genetically modified silkworm or cell comprising at least one edited chromosomal sequence. In particular, the chromosomal sequence is edited using a zinc finger nuclease-mediated editing process. The disclosure also provides zinc finger nucleases that target specific chromosomal sequences in the silkworm genome.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified silkworm or silkworm cells comprising at least one edited chromosomal sequence. In particular, the invention relates to the use of targeted zinc finger nucleases to edit chromosomal sequences in the silkworm.

### BACKGROUND OF THE INVENTION

The silkworm, Bombyx mori, is one of the most important insects economically, being employed in the production of silk—sericulture. Around the world, 70 million pounds of raw silk is produced every year. The production value of China's silk industry in 2006 reached 17.7 billion dollars. Although man-made fibers such as polyester, nylon, and acetate have replaced silk in many instances, many of the qualities of silk cannot be reproduced. Silk is highly valued because it possesses many excellent properties. Not only the luxurious look and feel of silk, but it is also lightweight, resilient, and extremely strong—one filament of silk is stronger then a comparable filament of steel. In addition, silk is a naturally self adjusting fabric, cool in the summer and warm in the winter.

From sericulture's standpoint, the manufacturing processes are complicated after cocoons are formed. First, either gas or boiling water is used to kill the silkworm, and then the silk fiber needs to be unraveled and degummed to get rid of sericin. Subsequent processes include cleaning, bleaching, dyeing, weighting, and lastly, finishing. In some occasions, silk producers allow the moths to emerge from the cocoon and then salvage the damaged cocoons. Because of the more humane harvesting of these silk cocoons, this silk is often called peace silk or vegetarian silk. However, peace silk is often slightly discolored by the alkaline solution secreted by the moth to create the hole, and it is not as strong and has a slightly different look and feel than conventional Bombyx mori silk. The complexity of the manufacturing processes and the desire to reduce the discoloration of peace silk require innovative solutions.

Scientifically, silkworms have occasioned important research activities. Because of silkworm's large size, complex metabolism and the abundance of mutants, it is also one of the best-characterized models for biochemical, molecular genetic and genomic studies of the order Lepidoptera. Recently, over 150,000 expressed sequence tags (ESTs) for silkworm have been sequenced, and the draft genome sequences, derived from the silkworm strains Dazao and p50, respectively, have been finished. To date, the BGI Gene Finder program predicted that the silkworm genome encodes roughly 18,510 genes. A Bombyx mori Microarray Database (BmMDB) and web-browser to store the silkworm gene expression data has been constructed and open to the public. In addition, as early as 1905, genetic hybrids of silkworms were bred for improved vigor and silk production. Today with more than two hundred mutations mapped, the silkworm is well established as a molecular genetic model for solving a broad range of fundamental biological problems.

The understanding of the silkworm gene profile and function and the capability to edit the genomic sequences enable the creation of transgenic silkworms. For example, transgenic silkworms can increase the silk production and disease resistance, obtain desirable features in the product and for the manufacturing processes, and be used as bioreactors for the production of proteinaceous drugs and specific biomaterials. In addition, there are needs to improve the quality of peace silk, utilize the wild silk, reduce allergy, enhance work-place safety, improve the manufacturing efficiency, reduce environmental impact from the manufacturing process and increase the silk strength for national security use. Therefore, there is a need for improved methods of editing genes coding firbroin and sericin proteins in silkworms, as well as means of modifying genes involved in desirable silkworm phenotypes in different species.

### SUMMARY OF THE INVENTION

One aspect of the present disclosure encompasses a genetically modified silkworm comprising at least one edited chromosomal sequence.

Another aspect provides a cell or cell line derived from a genetically modified silkworm comprising at least edited chromosomal sequence.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a protein associated with silkworm diseases or traits. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with silkworm diseases or traits generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with silkworm diseases or traits using targeted zinc finger nuclease technology is rapid, precise, and highly efficient.

### (I) Genetically Modified Silkworm

One aspect of the present disclosure provides a genetically modified silkworm in which at least one chromosomal sequence encoding a disease- or trait-related protein has been edited. For example, the edited chromosomal sequence may be inactivated such that the sequence is not transcribed and/or a functional disease- or trait-related protein is not produced. Alternatively, the edited chromosomal sequence may be modified such that it codes for an altered disease- or trait-related protein. For example, the chromosomal sequence may be modified such that at least one nucleotide is changed and the expressed disease- or trait-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed disease- or trait-related protein comprises a single amino acid deletion or insertion, provided such a protein is functional. For example, a protein coding sequence may be inactivated such that the protein is not produced. Alternatively, a microRNA coding sequence may be inactivated such that the microRNA is not produced. Furthermore, a control sequence may be inactivated such that it no longer functions as a control sequence. The modified protein may have altered substrate specificity, altered enzyme activity, altered kinetic rates, and so forth. Furthermore, the edited chromosomal sequence may comprise an integrated sequence and/or a sequence encoding an orthologous protein associated with a disease or a trait. The genetically modified silkworm disclosed herein may be heterozygous for the edited chromosomal sequence encoding a protein associated with a disease or a trait. Alternatively, the genetically modified silkworm may be homozygous for the edited chromosomal sequence encoding a protein associated with a disease or a trait.

In one embodiment, the genetically modified silkworm may comprise at least one inactivated chromosomal sequence encoding a disease- or trait-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional disease- or trait-related protein is not produced. The inactivated chromosomal sequence comprises no exogenously introduced sequence. Such a silkworm may be termed a “knockout.” Also included herein are genetically modified silkworms in which two, three, four, five, six, seven, eight, nine, or ten or more chromosomal sequences encoding proteins associated with a disease or a trait are inactivated.

In another embodiment, the genetically modified silkworm may comprise at least one edited chromosomal sequence encoding an orthologous protein associated with a disease. The edited chromosomal sequence encoding an orthologous disease- or trait-related protein may be modified such that it codes for an altered protein. For example, the edited chromosomal sequence encoding a disease- or trait-related protein may comprise at least one modification such that an altered version of the protein is produced. In some embodiments, the edited chromosomal sequence comprises at least one modification such that the altered version of the disease-related protein results in the disease in the silkworm. In other embodiments, the edited chromosomal sequence encoding a disease- or trait-related protein comprises at least one modification such that the altered version of the protein protects against a disease or does not form a trait in the silkworm. The modification may be a missense mutation in which substitution of one nucleotide for another nucleotide changes the identity of the coded amino acid.

In yet another embodiment, the genetically modified silkworm may comprise at least one chromosomally integrated sequence. The chromosomally integrated sequence may encode an orthologous disease- or trait-related protein, an endogenous disease- or trait-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a silkworm disease- or trait-related protein may be integrated into a “safe harbor” locus, such as the Rosa26 locus, HPRT locus, or AAV locus. In one iteration of the disclosure an animal comprising a chromosomally integrated sequence encoding disease- or trait-related protein may be called a “knock-in”, and it should be understood that in such an iteration of the animal, no selectable marker is present. An animal comprising a chromosomally integrated sequence encoding a silkworm or human disease, or trait-related protein may be called a “knock-in.” The present disclosure also encompasses genetically modified animals in which two, three, four, five, six, seven, eight, nine, or ten or more sequences encoding protein(s) associated with a disease or a trait are integrated into the genome.

In an exemplary embodiment, the genetically modified silkworm may be a “humanized” silkworm comprising at least one chromosomally integrated sequence encoding a functional human disease or trait-related protein. The functional human disease or trait-related protein may have no corresponding ortholog in the genetically modified silkworm. Alternatively, the wild-type silkworm from which the genetically modified silkworm is derived may comprise an ortholog corresponding to the functional human disease or trait-related protein. In this case, the orthologous sequence in the “humanized” silkworm is inactivated such that no functional protein is made and the “humanized” silkworm comprises at least one chromosomally integrated sequence encoding the human disease or trait-related protein. Those of skill in the art appreciate that “humanized” silkworms may be generated by crossing a knock out silkworm with a knock in silkworm comprising the chromosomally integrated sequence.

The chromosomally integrated sequence encoding a disease or trait-related protein may encode the wild type form of the protein. Alternatively, the chromosomally integrated sequence encoding a disease- or trait-related protein may comprise at least one modification such that an altered version of the protein is produced. In some embodiments, the chromosomally integrated sequence encoding a disease or trait-related protein comprises at least one modification such that the altered version of the protein produced causes a disease or forms a trait. In other embodiments, the chromosomally integrated sequence encoding a disease- or trait-related protein comprises at least one modification such that the altered version of the protein protects against the development of a disease or an undesirable trait.

In yet another embodiment, the genetically modified silkworm may comprise at least one edited chromosomal sequence encoding a disease or trait-related protein such that the expression pattern of the protein is altered. For example, regulatory regions controlling the expression of the protein, such as a promoter or transcription binding site, may be altered such that the disease or trait-related protein is over-produced, or the tissue-specific or temporal expression of the protein is altered, or a combination thereof. Alternatively, the expression pattern of the disease or trait-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a disease or trait-related protein. The genetically modified silkworm comprising the lox-flanked chromosomal sequence encoding a disease or trait-related protein may then be crossed with another genetically modified silkworm expressing Cre recombinase. Progeny comprising the lox-flanked chromosomal sequence and the Cre recombinase are then produced, and the lox-flanked chromosomal sequence encoding a disease or trait-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a disease or trait-related protein.

Exemplary examples of silkworm chromosomal sequences to be edited include those that code for proteins specifically expressed in silk gland. The silk gland is the site where silk proteins are synthesized and can be divided into three morphologically and functionally distinct compartments: ASG, MSG and PSG. In one embodiment, the genetically modified silkworm comprising modified silk fibroin proteins in PSG, including fibroin heavy chain (FibH), fibroin light chain (FibL) and fibrohexamerin P25 may have silk fiber of different phenotype in color, texture, smoothness, length, strength, weight or the ability to absorb dyes. In other embodiments, the genetically modified silkworm comprises a modified gene encoding the juvenile hormone binding protein which is involved in juvenile hormone signal transduction in the PSG and mediating the growth and development of the silk gland. Yet in another embodiment, the genetically modified silkworm comprises a modified ser1 gene in the MSG, which yields the glue protein sericin that is sticky and coats the outside of the silk strand over the fibroin protein core. Sericin comprises about 10-25% of silk, and has to be degummed during the silk processing. Genetically modified silkworm comprising a modified ser1 gene may produce silk fiber without the need of extensive degumming process. As a result, genetically modified silk fiber doesn't need the “weighting” practice by adding metals to silk fabric in textile manufacturing.

A non-limiting example of a group of proteins involved in silk production is transporters involved in transporting substances relative to silk formation, such as members of the solute carrier family (family 35 member B3, member E1, and family 39 member 9) and the transmembrane trafficking protein isoform 2. Those of skill in the art appreciate that other proteins are involved in silk color, texture, smoothness, uniformity, length, strength, weight and the ability to absorb dyes, but the genetic loci have not been determined.

Protease inhibitors in A/MSG may play an important role in protecting the fibroin proteins in the silk gland lumen against digestion by proteases, such as antennal esterase and serine protease, which are expressed in the A/MSG. In another embodiment, the genetically modified silkworm may comprise an edited chromosomal sequence encoding protease inhibitor in A/MSG, wherein the edited chromosomal sequence comprises at least one modification such that an altered version of protease inhibitor is produced. The modification may be a missense mutation in which substitution of one nucleotide for another nucleotide changes the identity of the coded amino acid. The protease inhibitor coding region may be edited to comprise more than one missense mutation such that more than one amino acid is changed. Additionally, the chromosomal region may be modified to have a three nucleotide deletion or insertion such that the expressed protease inhibitor protein comprises a single amino acid deletion or insertion, provided such a protein is functional. Those of skill in the art will appreciate that many different modifications are possible in the protease inhibitor coding region. The modified protease inhibitor coding region may give rise to a silk protein with different physical properties. In one embodiment, the genetically modified silkworm comprising a modified protease inhibitor chromosomal region may have a phenotype producing silk without high percentage of sericin yet still intact in shape and other physical properties.

In still another embodiment, the genetically modified silkworm may comprise an edited chromosomal sequence encoding Fibroin, Sericin, solute carrier, protease inhibitor or combinations thereof. The edited chromosomal sequence may comprise at least one modification such that an altered version of Fibroin, Sericin or other silk fiber formation related proteins is produced. The chromosomal sequence may be modified to contain at least one nucleotide change such that at the expressed protein comprises at least one amino acid change as detailed above. Alternatively, the edited chromosomal sequence may comprise a mutation such that the sequence is inactivated and no protein is made or a defective protein is made. As detailed above, the mutation may comprise a deletion, an insertion, or a point mutation. The genetically modified silkworm comprising an edited FibH, ser1 and/or protease inhibitor chromosomal sequence may have a different fiber color, texture, weight than a silkworm in which said chromosomal region(s) is not edited.

Silk is naturally hypoallergenic. However, several people experience silk allergies for a wide variety of causes. Often, the allergies are traced to the diet of the silk worm, such as mulberry or oak leaves, which influence the protein chains found in the silk strands produced by the silkworm. Silk allergies can cause asthma or allergic rhinitis. In one embodiment, the genetically modified silkworm may comprise an edited chromosomal sequence encoding alpha and beta glucosidases, glycoside hydrolase, and glucose transporters are all involved in glucose hydrolysis and transport in the digestion of mulberry leaves, the sole food source for the silkworm. These proteins are expressed in midgut of silkworm and are related to functions such as nutrient digestion, transportation, and absorption. In another embodiment, the genetically modified silkworm may comprise an edited chromosomal sequence encoding the lipase protein family, antennal esterases, carboxylesterase, and scavenger receptor SR-B1, which are associated mainly with lipid metabolism in midgut, such as the hydrolysis of triglycerides, degradation of odorant acetate compounds, and the binding of modified low-density lipoproteins. The genetically modified silkworm comprising the edited chromosomal sequence described above generally will not contain allergen, which causes silk allergic reactions for silk manufacturing workers and consumers.

The midgut also represents the first line of resistance and immune response of the silkworm. In one embodiment, the genetically modified silkworm may comprise an edited chromosomal sequence encoding aminopeptidases which bind various classes of the Cry toxins. For example, BtR175, a cadherin-like protein expressed in the silkworm, functions as a Cry toxin receptor in signal transduction. In another embodiment, the genetically modified silkworm may comprise an edited chromosomal sequence of 17 members of the cytochrome P450 gene family in the midgut, which include CYP4, CYP6 and CYP9. The cytochrome P450 gene family in the midgut is involved in metabolism of plant toxins and insecticides.

Another midgut-specific gene encodes peptidoglycan recognition protein, which can bind strongly to the cell wall peptidoglycans of Gram-positive bacteria and trigger the immune response. Furthermore, two lymphocyte receptor genes expressed specifically in the midgut encode binding proteins that function in the recognition of pathogens. In another group of embodiments, the genetically modified silkworm may comprise an edited chromosomal sequence in peptidoglycan recognition protein or lymphocyte binding protein, wherein the chromosomal sequence is up-regulated such that the silkworm is more disease resistant. With suitable mutations discussed above, the genetically modified silkworm generally will have better immune system, is disease- and pathogen-free, and is less susceptible to plant toxins and insecticides in its food source.

Similar to silkworm fiber, spider silk is another naturally made fiber which is three times tougher than Kevlar®, a material used in the army's current ballistic protective vest. Spider silk's superior ability to elongate allows it to absorb more energy in breaking and slow down of a projectile more effectively. However, the strong, pliable silk that spiders produce is not practical to harvest. The gene to make spider silk from the spider N. clavipes has been cloned, and there are also synthetic genes to mimic the spider dragline silk. In one embodiment, the genetically modified silkworm may comprise an edited chromosomal sequence that comprises an integrated sequence, such as flagelliform gene, coding spider silk and alike. The genetically modified silkworm will enable a systematic and high-volume production of spider silk for the need of new material with unique properties.

The present disclosure also encompasses a genetically modified silkworm comprising any combination of the above described chromosomal alterations. For example, the genetically modified silkworm may comprise a modified FibH and/or FibL chromosomal sequence, a modified ser1 chromosomal sequence, and/or a modified BtR175, and/or CYP4 chromosomal sequence, and/or integrated sequence from other species or organisms.

Additionally, the silkworm disease- or trait-related gene may be modified to include a tag or reporter gene, as is well-known. Reporter genes include those encoding selectable markers such as cloramphenicol acetyltransferase (CAT) and neomycin phosphotransferase (neo), and those encoding a fluorescent protein such as green fuorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

The genetically modified silkworm may be heterozygous for the edited chromosomal sequence or sequences. In other embodiments, the genetically modified silkworm may be homozygous for the edited chromosomal sequence or sequences.

Typically, the silkworm will be from the genus Bombyx mori. However, the genetically modified silkworm may be a member of genera that are known to individuals skilled in the art. As used herein, the term “silkworm,” encompasses egg, embryos, cocoon, moth and organisms of silkworm at various developmental stages. In each of the foregoing iterations of suitable silkworms for the invention, the silkworm does not include exogenously introduced, randomly integrated transposon sequences.

### (II) Genetically Modified Silkworm Cells

A further aspect of the present disclosure provides genetically modified silkworm cells or cell lines comprising at least one edited chromosomal sequence. The disclosure also encompasses a lysate of said cells or cell lines. The genetically modified silkworm cell (or cell line) may be derived from any of the genetically modified silkworms disclosed herein. Alternatively, the chromosomal sequence may be edited in a silkworm cell as detailed below.

The silkworm cell may be any established cell line or a primary cell line that is not yet described. The cell line may be adherent or non-adherent, or the cell line may be grown under conditions that encourage adherent, non-adherent or organotypic growth using standard techniques known to individuals skilled in the art. The silkworm cell or cell line may be derived from egg, cocoon, moth, or organs from silkworm at different developmental stages, and so forth. Additionally, the silkworm cell or cell line may be a silkworm stem cell. Suitable stem cells include without limit embryonic stem cells, ES-like stem cells, adult stem cells, pluripotent stem cells, induced pluripotent stem cells, multipotent stem cells, oligopotent stem cells, and unipotent stem cells.

Similar to the genetically modified silkworms, the genetically modified silkworm cells may be heterozygous or homozygous for the edited chromosomal sequence or sequences.

### (III) Zinc Finger-Mediated Genome Editing

In general, the genetically modified silkworm or silkworm cell, as detailed above in sections (I) and (II), respectively, is generated using a zinc finger nuclease-mediated genomic editing process. The process for editing a silkworm chromosomal sequence comprises: (a) introducing into a silkworm embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration, the sequence flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. The embryo used in the above described method typically is a fertilized one-cell stage embryo.

Components of the zinc finger nuclease-mediated method of genome editing are described in more detail below.

### (a) Zinc Finger Nuclease

The method comprises, in part, introducing into a silkworm embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA is known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger DNA binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition regions may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:I538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Exchange Polynucleotide

The method for editing chromosomal sequences may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may be at least about 80, 81, 82, 83, 84, 85, 86, 87, 88, 89, 90, 91, 92, 93, 94, 95, 96, 97, 98, or 99% identical a region of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (c) Optional Donor Polynucleotide

The method for editing chromosomal sequences may further comprise introducing at least one donor polynucleotide comprising a sequence for integration into the embryo or cell. A donor polynucleotide comprises at least three components: the sequence to be integrated that is flanked by an upstream sequence and a downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide may be a DNA plasmid.

The donor polynucleotide comprises a sequence for integration. The sequence for integration may be a sequence endogenous to the silkworm or it may be an exogenous sequence. Additionally, the sequence to be integrated may be operably linked to an appropriate control sequence or sequences. The size of the sequence to be integrated can and will vary. In general, the sequence to be integrated may range from about one nucleotide to several million nucleotides.

The donor polynucleotide also comprises upstream and downstream sequence flanking the sequence to be integrated. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for editing a chromosomal sequence by integrating a sequence, the double stranded break introduced into the chromosomal sequence by the zinc finger nuclease is repaired, via homologous recombination with the donor polynucleotide, such that the sequence is integrated into the chromosome. The presence of a double-stranded break facilitates integration of the sequence. A donor polynucleotide may be physically integrated or, alternatively, the donor polynucleotide may be used as a template for repair of the break, resulting in the introduction of the sequence as well as all or part of the upstream and downstream sequences of the donor polynucleotide into the chromosome. Thus, the endogenous chromosomal sequence may be converted to the sequence of the donor polynucleotide.

### (d) Delivery of Nucleic Acids

To mediate zinc finger nuclease genome editing, at least one nucleic acid molecule encoding a zinc finger nuclease and, optionally, at least one exchange polynucleotide or at least one donor polynucleotide is delivered into the silkworm embryo or cell. Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and an exchange (or donor) polynucleotide are introduced into an embryo or cell, the ratio of exchange (or donor) polynucleotide to nucleic acid encoding a zinc finger nuclease may range from about 1:10 to about 10:1. In various embodiments, the ratio of exchange (or donor) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one exchange (or donor) polynucleotide is introduced into an embryo or cell, the nucleic acids may be introduced simultaneously or sequentially. For example, nucleic acids encoding the zinc finger nucleases, each specific for a distinct recognition sequence, as well as the optional exchange (or donor) polynucleotides, may be introduced at the same time. Alternatively, each nucleic acid encoding a zinc finger nuclease, as well as the optional exchange (or donor) polynucleotides, may be introduced sequentially.

### (e) Culturing the Embryo or Cell

The method for editing a chromosomal sequence using a zinc finger nuclease-mediated process further comprises culturing the silkworm egg embryo or cell comprising the introduced nucleic acid(s) to allow expression of the zinc finger nuclease.

A silkworm egg embryo may be cultured in vitro (e.g., in cell culture). Typically, the silkworm egg embryo is cultured for a short period of time at an appropriate temperature and in appropriate media with the necessary O₂/CO₂ ratio to allow the expression of the zinc finger nuclease. A skilled artisan will appreciate that culture conditions can and will vary depending on the silkworm species. Routine optimization may be used, in all cases, to determine the best culture conditions for a genetically modified silkworm egg embryo. In some cases, a cell line may be derived from an in vitro-cultured embryo (e.g., an embryonic stem cell line).

Similarly, cells comprising the introduced nucleic acids may be cultured using standard procedures to allow expression of the zinc finger nuclease. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no exchange (or donor) polynucleotide, the zinc finger nuclease recognizes, binds, and cleaves the target sequence in the chromosomal sequence of interest. The double-stranded break introduced by the zinc finger nuclease is repaired by the error-prone non-homologous end-joining DNA repair pathway. Consequently, a deletion, insertion, or nonsense mutation may be introduced in the chromosomal sequence such that the sequence is inactivated.

In cases in which the embryo or cell comprises an expressed zinc finger nuclease as well as an exchange (or donor) polynucleotide, the zinc finger nuclease recognizes, binds, and cleaves the target sequence in the chromosome. The double-stranded break introduced by the zinc finger nuclease is repaired, via homologous recombination with the exchange (or donor) polynucleotide, such that a portion of the chromosomal sequence is converted to the sequence in the exchange polynucleotide or the sequence in the donor polynucleotide is integrated into the chromosomal sequence. As a consequence, the chromosomal sequence is modified.

The genetically modified silkworms disclosed herein may be crossbred to create silkworms comprising more than one edited chromosomal sequence or to create silkworms that are homozygous for one or more edited chromosomal sequences. Those of skill in the art will appreciate that many combinations are possible. Moreover, the genetically modified silkworms disclosed herein may be crossbred with other silkworms to combine the edited chromosomal sequence with other genetic backgrounds. By way of non-limiting example, suitable genetic backgrounds may include wild-type, natural mutations giving rise to known silkworm phenotypes, targeted chromosomal integration, non-targeted integrations, etc.

### (IV) Applications

The silkworms and cells disclosed herein may have several applications. In one embodiment, the genetically modified silkworm comprising at least one edited chromosomal sequence may exhibit a phenotype desired by humans. For example, modification of the chromosomal sequence encoding Fibroin may result in silk fiber that carries unique color, texture, weight or strength. In other embodiments, the silkworm comprising at least one edited chromosomal sequence may be used as a model to study the genetics of silk composition, production, and/or transportation. Additionally, a silkworm comprising at least one disrupted chromosomal sequence may be used as a model to study a disease or condition that affects humans or other animals. Non-limiting examples of suitable diseases or conditions include mulberry allergy. Additionally, the disclosed silkworm cells and lysates of said cells may be used for similar research purposes.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor or exchange polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of FibH in Model Organism Cells

Zinc finger nuclease (ZFN)-mediated genome editing may be tested in the cells of a model organism such as silkworm, Bombyx mori, using a ZFN that binds to the chromosomal sequence of a silkworm fiber related gene of the silkworm cell such as Fibroin heavy chain (FibH), Fibroin light chain (FibL), fibrohexamerin P25, Sericin (Ser1), Cry toxin receptor (BtR175), Cytochrome P450 (CYP4, CYP6, CYP9). The particular silk fiber-related gene to be edited may be a gene having identical DNA binding sites to the DNA binding sites of the corresponding insect, such as spider homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into silkworm, Bombyx mori, cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products may be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected cognition-related gene locus in silkworm, Bombyx mori, cells using a ZFN.

### Example 2

### Genome Editing of FibH in Model Organism Embryos

The embryos of a model organism such as silkworm, Bombyx mori, egg embryo may be harvested using standard procedures and injected with capped, polyadenylated mRNA encoding a ZFN similar to that described in Example 1. The silkworm, Bombyx mori, egg embryos may be at the one cell stage when microinjected. Control embryos were injected with 0.1 mM EDTA. The frequency of ZFN-induced double strand chromosomal breaks may be estimated using the Cel-1 assay as described in Example 1. The cutting efficiency may be estimated using the CEI-1 assay results.

The development of the embryos following microinjection may be assessed. Embryos injected with a small volume ZFN mRNA may be compared to embryos injected with EDTA to determine the effect of the ZFN mRNA on embryo survival to later stage.

What is claimed is:

1. A genetically modified silkworm comprising at least one edited chromosomal sequence.

2. The genetically modified silkworm of claim 1, wherein the edited chromosomal sequence is inactivated, is modified, or has an integrated sequence.

3. The genetically modified silkworm of claim 1, wherein the edited chromosomal sequence encodes a protein chosen from Fibroin heavy chain (FibH), Fibroin light chain (FibL), fibrohexamerin P25, Sericin (Ser1), Cry toxin receptor (BtR175), Cytochrome P450 (CYP4, CYP6, CYP9), and combinations thereof.

4. The genetically modified silkworm of claim 3, wherein the protein is FibH, and the edited chromosomal sequence comprises at least one mutation such that the sequence is modified and the expressed protein comprises at least one amino acid change.

5. The genetically modified silkworm of claim 4, wherein the silkworm produces silk with different color, composition, texture, strength, weight or dye absorbance.

6. The genetically modified silkworm of claim 3, wherein the protein is FibL, and the edited chromosomal sequence comprises at least one mutation such that the sequence is modified and the expressed protein comprises at least one amino acid change.

7. The genetically modified silkworm of claim 6, wherein the silkworm produces silk of a different color, composition, texture, strength, weight or dye absorbance than a silkworm in which the chromosomal region is not edited.

8. The genetically modified silkworm of claim 3, wherein the protein is fibrohexamerin P25 or Sericin (Ser1), and the edited chromosomal sequence comprises at least one mutation such that the sequence is modified.

9. The genetically modified silkworm of claim 8, wherein the silkworm produces silk of a different color, composition, texture, strength, weight or dye absorbance than a silkworm in which the chromosomal region is not edited.

10. The genetically modified silkworm of claim 3, wherein the protein is Cry toxin receptor (BtR175), and the edited chromosomal sequence comprises at least one mutation such that the sequence is modified and the expressed protein comprises at least one amino acid change.

11. The genetically modified silkworm of claim 10, wherein the silkworm produces silk of a different phenotype than a silkworm in which the chromosomal sequence is not edited.

12. The genetically modified silkworm of claim 3, wherein the protein is Cytochrome P450 (CYP4, CYP6, CYP9), and the edited chromosomal sequence comprises at least one mutation such that the sequence is modified and the expressed protein comprises at least one amino acid change.

13. The genetically modified silkworm of claim 12, wherein the silkworm produces silk of a different phenotype than a silkworm in which the chromosomal sequence is not edited.

14. The genetically modified silkworm of claim 1, wherein the edited chromosomal sequence encodes a protein that comprises an integrated sequence chosen from spider flagelliform gene and other spider silk formation genes, and combinations thereof.

15. The genetically modified silkworm of claim 14, wherein the integrated sequence is spider flagelliform gene, and the edited silkworm chromosomal sequence comprises the integrated sequence such that the silkworm chromosomal sequence is modified and the expressed protein comprises at least one amino acid change.

16. The genetically modified silkworm of claim 15, wherein the silkworm produces silk similar to spider silk in its strength or pliability.

17. The genetically modified silkworm of claim 1, wherein the silkworm is heterozygous or homozygous for the edited chromosomal sequence.

18. The genetically modified silkworm of claim 1, wherein the silkworm is a domesticated.

19. The genetically modified silkworm of claim 1, wherein the silkworm is an embryo, an egg, a worm, a cocoon, or a moth.

20. A silkworm egg embryo, the embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

21. The silkworm egg embryo of claim 20, wherein the chromosomal sequence encodes a protein chosen from Fibroin heavy chain (FibH), Fibroin light chain (FibL), fibrohexamerin P25, Sericin (Ser1), Cry toxin receptor (BtR175), Cytochrome P450 (CYP4, CYP6, CYP9).

22. A genetically modified silkworm cell comprising at least one edited chromosomal sequence.

23. The genetically modified silkworm cell of claim 22, wherein the edited chromosomal sequence encodes a protein chosen from Fibroin heavy chain (FibH), Fibroin light chain (FibL), fibrohexamerin P25, Sericin (Ser1), Cry toxin receptor (BtR175), Cytochrome P450 (CYP4, CYP6, CYP9), and combinations thereof.
